(19) United States
(12) Patent Application Publication
(10) Pub. No.: US 2016/0235497 A1
(43) Pub. Date: Aug. 18, 2016

(54) Blood Pressure Cuff Cover with Rip-Away Liners.

(71) Applicant: Dominic Javier, El Cajon, CA (US)

(72) Inventor: Dominic Javier, El Cajon, CA (US)

(21) Appl. No.: 15/043,523

(22) Filed: Feb. 13, 2016

Related U.S. Application Data
(63) Continuation of application No. 62/116,745, filed on Feb. 16, 2015.

(51) Int. Cl. A61B 46/10; A61B 5/022

(52) U.S. Cl. CPC A61B 46/10; A61B 5/022; A61B 2046/205

(57) ABSTRACT

A cover for a blood pressure cuff is disclosed. A base portion with tabs removably attaches to the blood pressure cuff. The base portion has attached to it a stack of sheets. The stack features multiple sheets of thin material where each sheet can be ripped off the stack after the blood pressure cuff has been used to take a patient's blood pressure. Sequential numbers on each sheet allow for tracking of use patterns.

### CROSS REFERENCE TO RELATED APPLICATIONS

This application claims priority back to U.S. Provisional Application No. 62/116,745, filed Feb. 16, 2015, a copy of which is attached to this application and the contents of which are incorporate by reference.

### STATEMENT REGARDING FEDERALLY SPONSORED RESEARCH OR DEVELOPMENT

This invention was not federally sponsored.

### BACKGROUND OF THE INVENTION

1. Field of the Invention

This invention relates to the general field of medical devices, and more specifically toward sanitary coverings of medical devices used repetitively on humans. To this end, a blood pressure cover is provided which has numerous rip-away sheets of protective film that can be used to take the blood pressure of a patient and then quickly rip off the sheet and discard it, leaving a new, sanitized sheet in place for the next patient.

2. History of the Field of Invention

One of the most common concerns in a hospital or other medical facility is the sanitation of the medical instruments. Invasive tools such as surgeon's knives and scalpels are thoroughly washed and sanitized after every use to make sure that the germs from the first patient are not carried by the tools to successive patients. Bedding from hospital beds is washed thoroughly between uses, and examination tables usually have disposable covers that are thrown out after every patient. Other medical devices such as needles, otoscope specular, tongue depressors, and bandages are generally thrown out after a single use.

There are, however, some medical devices that are used just as frequently that are rarely cleaned and have the opportunity to pass on germs from one patient to the others. One of the best known of these devices is the blood pressure cuff. Blood pressure cuffs have several basic parts. There is a cuff, which is a relatively flat, rectangular piece that is wrapped around a patient's arm. The cuff has an internal bladder that can be filled with air, one or more tubes connecting the internal bladder to a bulb, or source of air pressure, and one or more gauges to measure the blood pressure as the technician inflates and deflates the bladder to measure the systolic and diastolic blood pressures.

Since one of the first things a check-in nurse will do is measure the blood pressure of a patient, the average blood pressure cuff may be used 50 or more times each day. These cuffs have no mechanism by which they can be sanitized other than by washing them, which can be dangerous as they have rubber and metal parts that may be damaged by water and/or high temperatures. In addition, the most common method of sanitizing blood pressure cuffs in healthcare facilities currently is to use disinfectant wipes (such as “pdi super sani cloth”) that contain quaternary ammonium compounds, which requires a minimum of three minutes “wet time” for porous surfaces such as blood pressure cuffs to be effective. If you include the drying time, it will take up at least five minutes of the healthcare worker's time, and adding a potential five minutes to the turn-around time for each patient is simply not practical. There is currently no system for monitoring whether clinicians are indeed using the wipes to sanitize blood pressure cuffs, and because of the time needed to do it effectively, this method is rarely used. As with any medical device that comes in contact with thousands of patients before being disposed of, there is significant risk that a patient with some sort of infection could infect subsequent users upon which the same blood pressure cuff was used. However, since it will take the clinician more time to clean and dry the blood pressure cuff than it takes to actually take the blood pressure, blood pressure cuffs are rarely cleaned, and most certainly are not routinely cleaned between each patient.

Thus there has existed a long-felt need for a blood pressure cuff that does not expose the permanent parts of the cuff to human skin. Toward solving this need, the current invention has a sheet of tear-off films arrayed on a cover that is removably attached to the blood pressure cuff such that the outer film can be quickly, easily, and economically torn off after a single use, thereby leaving the blood pressure cuff (clean and sanitized) with a new, clean contact surface for the next patient.

### SUMMARY OF THE INVENTION

The current invention provides just such a solution by having a means by which a clinician taking the blood pressure of multiple patients can provide each patient with a clean, sterile portion of the cuff to cover their skin.

It is a principal object of the invention to provide a removable stack of thin film sheets that can protect the skin of a patient from any germs contained on the blood pressure cuff.

It is another object of the invention to provide a way a clinician can (treat) take blood pressure measurements of a succession of patients without having to clean the blood pressure cuff or replace any part of it.

Another object of the invention is to provide a system by which a different prophylactic sheet can be used to protect the skin of each in a series of patients, and the removal of the used sheet is so easy and quick that the clinicians will actually use the invention, as opposed to the current state of the art where the time-intensive washing and drying is rarely done.

As a related object of the invention, the goal of making clinicians use the invention can be further accomplished by sequentially numbering the tear-away sheets such that a clinician who took blood pressure readings from 15 patients should show a decrease of 15 sheets on his or her stack.

It is a final object of this invention to provide an inexpensive, simple, and fast way to protect a series of patients from any germs or viruses contained in the skin of the previous patients.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention. It should be noted that there are both automatic and manual blood pressure monitors. The invention is illustrated here as fitting over an automatic blood pressure cuff, but the invention is equally applicable to a manual blood pressure measuring device.

FIG. 1 is a top view of one preferred embodiment of the invention, with a view of the base portion of the invention as initially manufactured and before the bends or crimps are put in to create the tabs that removably secure the invention to the blood pressure cuff. The base portion is used to grip the blood pressure cuff through the tabs, and the center flat portion is where the stack of thin, prophylactic sheets is attached.

FIG. 2 is a perspective view of the invention removably attached to a blood pressure cuff.

FIG. 3 is a perspective, exploded view of the current invention showing both the base portion and the rip-away sheets attached to a blood pressure cuff.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

### LIST OF REFERENCE NUMBERS

1. Base Portion
2. Tab
3. Non-adhesive portion of the Tab
4. Flat cover.
5. Adhesive section.
6. 6 through 19 intentionally left blank.
20. Blood pressure cuff fitted with base portion with rip-away films.
21. Tube/“Hook side” of the Velcro.
22. (Tube outlet) Tube
23. Valve/connector
24. Target
25. Range
26. Bottom sheet.
27 through 39 intentionally left blank.
40. Stack of rip-away sheets.
41. Numbers.
42. Uppermost sheet
43. Second sheet in stack

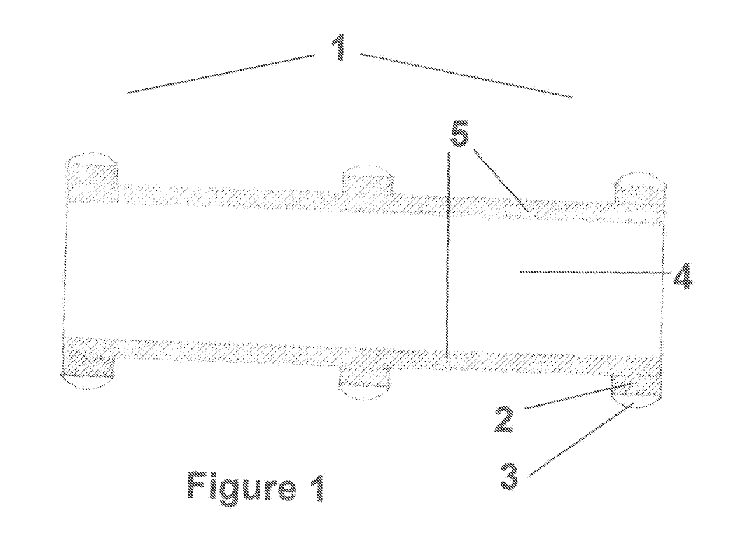

FIG. 1 is a top view of one preferred embodiment of the invention, with a view of the base portion, generally referenced as 1, of the invention as initially manufactured and before the bends or crimps are put in to create the tabs that removably secure the invention to the blood pressure cuff. The base portion is used to grip the blood pressure cuff through the tabs, and the center flat portion 4 is where the stack of thin, prophylactic sheets is attached. The tabs 2, have a non-adhesive portion 3 that can be used to removably adhere the base 1 to a blood pressure cuff.

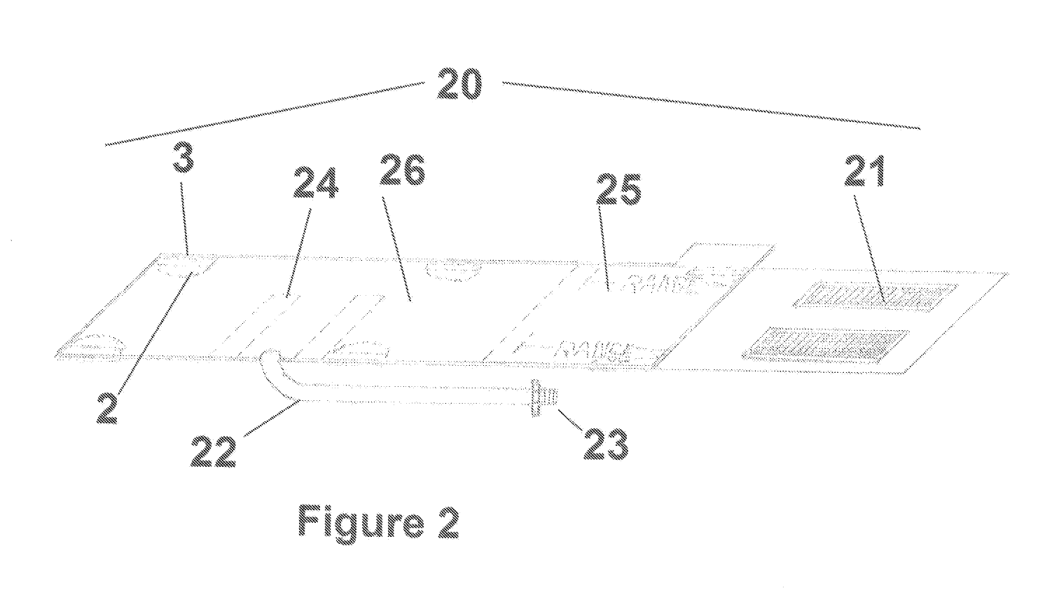

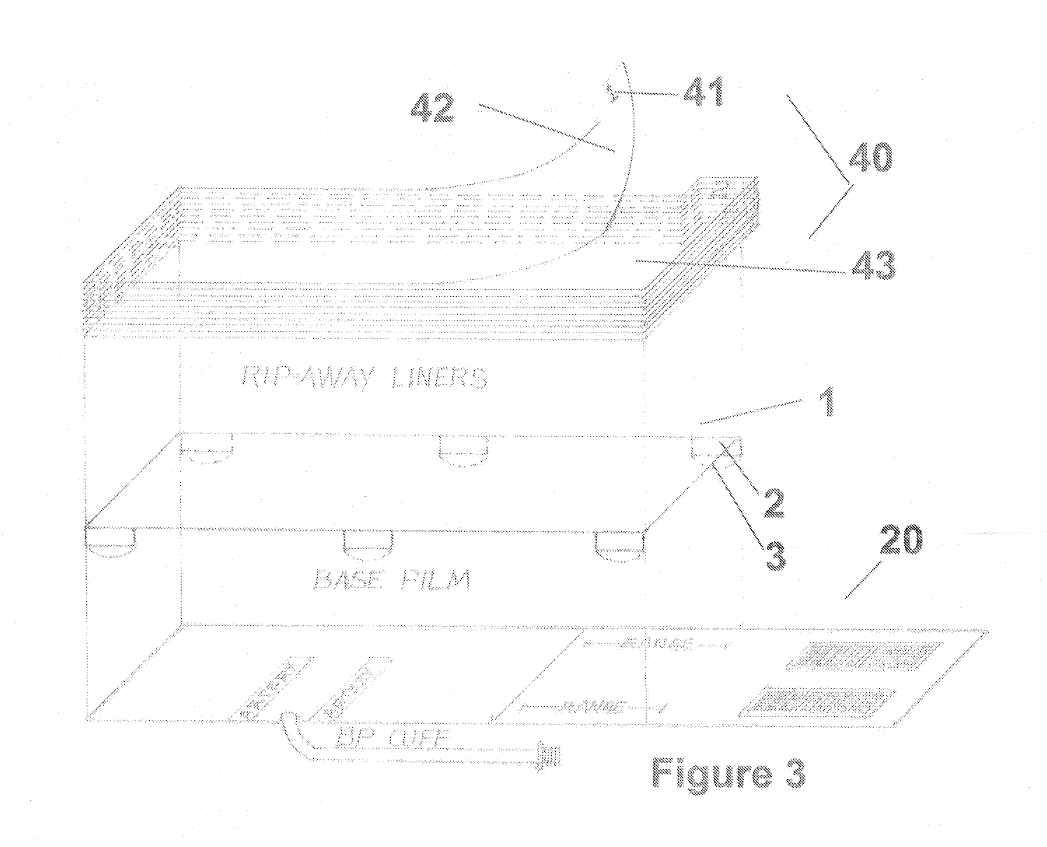

FIG. 2 is a perspective view of the invention removably attached to a blood pressure cuff. The bottom sheet 26 lies at the bottom of an entire stack of sheet (illustrated in FIG. 3) that lie on top of the blood pressure cuff, generally referenced as 20, held in place by the tabs 2, which in this figure have been wrapped completely over the blood pressure cuff to removably secure the invention to the blood pressure cuff. (The only portion of the blood pressure cuff that is not covered by the bottom sheet 26 is the attachment portion 21/) The bottom sheet 26 will cover the portion of the cuff from one end of the cuff up to the distal end of RANGE marker. The bottom sheet 26 and the rest of the sheets are wrapped around a patient's arm, and the blood pressure cuff secured by hook and loop or some other known means of attachment located on the backside of the blood pressure cuff. The range 25 indicates the size of a patient's arm upon which the invention can be used. The targets 24 indicate location of arteries to allow the user of the invention to correctly place it. A tube 22 from the blood pressure cuff can be attached through a (valve) connector 23 to a source of air pressure.

FIG. 3 is a perspective, exploded view of the current invention showing both the base portion and the rip-away sheets attached to a blood pressure cuff. A stack 40 of rip-away sheets is attached to a base 1. The base is removably fit over a blood pressure cuff 20 with tabs 2, which have a non-adhesive section 3 for easy attachment and removal. The stack 40 has a plurality of thin sheets that are sufficient to cover the skin of a patient such that any germs or viruses from the blood pressure cuff 20 do not touch the patient, and so that any germs or viruses that the patient is carrying do not, in turn, rub off on the blood pressure cuff 20 and infect subsequent users. After the device is used on the first patient, the tube 22 is kept to the side and the uppermost sheet 42 is ripped off and disposed of, leaving the second sheet 43 in the stack 40 for protecting the skin of the next patient. This assembly line continues until all the sheets are used up, whereupon the clinician merely removes the base 1 from the blood pressure cuff 20 and puts on another full stack 40.

Each sheet can be numbered 41 such that the number on the uppermost sheet 42 goes up (or down) by the count of one for every patient the clinician takes a blood pressure reading upon. This makes the clinicians more accountable for actually using the device as it was intended, and give a supervisor the ability to check up on clinicians to make sure they are providing the maximum protection to their patients from viruses, dirt and germs contained on the blood pressure cuff.

The base 1 has a flat cover 4 that covers a portion of the blood pressure cuff, and two or more tabs 2 that can be folded down to allow the base 1 to removably attach to the blood pressure cuff 20. Several versions of the tabs are contemplated. First, the base could be manufactured from rigid cardboard, such that when the tabs were bent over and around the blood pressure cuff, they would grip it with merely physical pressure. Second, the tab portions could have an adhesive substance 2 laid on them such that once the tab is folded over the blood pressure cuff it would stick. In either case, when the invention is in place, that portion of the blood pressure cuff that normally touches the skin, is covered by the stack of sheets and the patient is protected by the uppermost sheet 42. The Velcro® or other hook and loop fastener or other means of attachment 21 at the end of the blood pressure cuff wraps the invention tightly around the arm of the user, thereby ensuring that the patient's arm never touches the actual blood pressure cuff 20. In this particular figure, the tab 2 has a rectangular section that has its inside surface covered with a tacky but not permanent adhesive, and a semi-circular region 3 that is not covered with any adhesive. Thus, to remove the invention from a blood pressure cuff, the user need only grasp the non-adhesive portion 3 of the tab 2 and pull it off.

The design of the rip-away film is important, as the film is strong enough to avoid ripping when being used to encircle the arm of the patient, and yet thin enough so that a substantial number of sheets can be pressed, laminated, or otherwise pressed together to present a small profile and yet have enough sheets to maximize the time between having to replace the base. A number of mechanisms are contemplated to allow a user to grasp and tear off individual sheets. There could be a weak adhesive applied in between the individual sheets except for a corner, end or some sort of tab. The sheets could also have perforations at their rear section to allow for a clean tear-away. The rip-away sheets or film are also thin enough to allow a clinician to take accurate blood pressure readings even with the stack of sheets in place.

Individual sheets can be numbered 41, such that the number of sheets ripped off during a certain time period can be easily quantified. Thus, if the hospital directs its clinicians to tear off a single sheet after each patient, and the clinician begins a day's inventory on sheet #55, the supervisor could make periodic checks and after seeing ten patients, the uppermost sheet should be sheet #45. This would put the clinicians under pressure to use the invention, as opposed to the current art which allows a clinician to “clean” (or at least say they “cleaned”) blood pressure cuffs through applying a wet antiseptic cloth for three minutes and leaving it to dry for two additional minutes (which is why blood pressure cuffs are rarely cleaned at all, much less between each patient).

Other embodiments of the base portion are contemplated, including using hook-and-loop fasteners on the back of the base portion, or on a portion of the base portion, to attach it directly to the blood pressure cuff. It is also contemplated that the tabs could grip the blood pressure cuff through hook-and-loop fasteners, snaps, magnets and other known means of removable attachment.

The invention as described in this provisional patent application could be used for a blood pressure cuff that goes around the wrist, upper arm, or leg. While the upper arm version is by far the most popular, and therefore the one illustrated in the drawings, it should be understood that it is contemplated using this same technology on different types of blood pressure cuffs.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

That which is claimed:

1. A cover for a blood pressure cuff comprising
a stack of sheets and a base,
where the base comprises a flat portion of thin material, where the flat portion comprises a front, a back, and two side portions, and at least two tab portions, where the at least two tab portions comprise a section of thin material that extends laterally away from the side portions, where there is at least one tab on each of the two side portions, and where the at least two tab portions can be folded back on themselves of the edges of a blood pressure cuff to removably secure the blood pressure cuff cover to the blood pressure cuff;
where the stack of sheets comprises a plurality of thin, film members, where the stack of sheets additionally comprises a bottom sheet and an uppermost sheet, and a plurality of middle sheets, where the bottom sheet is attached to the flat portion of the base, and each of the middle sheets and the uppermost sheet is at least partially attached to the adjacent sheets, and each sheet in the stack of sheets can be easily detached from the stack of sheets;
whereby the uppermost sheet can be used to effectively protect a patient's arm from germs contained on the blood pressure cuff, and effectively prevent any germs on the patient's arm from contamination by the blood pressure cuff, then a clinician can easily and quickly remove the uppermost sheet from the stack, dispose of the uppermost sheet, and have a next sheet of the middle sheets be exposed as a new uppermost sheet, ready to protect the arm of the next patient.

2. The cover of claim 1, wherein the tabs additionally comprise an adhesive section and a plastic cover over the adhesive section that can be easily and quickly removed before removably attaching the cover to a blood pressure cuff.

3. The cover of claim 1, where the sheets additionally comprise a number.

4. The cover of claim 1, where each sheet additionally comprises a sequential number.

5. The cover of claim 1, where the stack of sheets additionally comprise a partial cut located at a back section of each sheet, such that a user of the invention can easily tear away individual sheets, where each sheet additionally comprises a base section and a corner section, where the base section additionally comprises an adhesive substance that allows the base section to removably adhere to a bottom sheet below the sheet, and the corner section lacks any such adhesive substance, thereby allowing a user of the invention to “tab” through the stack of sheets to select only the uppermost sheet for removal

6. The cover of claim 1, where the tabs additionally comprise an adhesive section and a plastic cover over the adhesive section that can be easily and quickly removed before removably attaching the cover to a blood pressure cuff.

7. A blood pressure cuff comprising
a main bladder and a cover;
where the cover comprises a stack of sheets and a base;
where the base comprises a flat portion of thin material and two tabs, where each tab comprises a section of thin material that extends away from the flat portion;
where the stack of sheets comprises a plurality of thin member sheets, where the plurality of sheets are removably secured together such that each sheet may easily detach from an adjacent sheet; and
where the cover is removably secured to the main bladder using the two tabs.

8. The blood pressure cuff of claim 7, where each tab of the base comprises an adhesive covering at least a portion of its surface, where the adhesive secures the tab to the main bladder.

9. The blood pressure cuff of claim 7, wherein each sheet of the stack of sheets comprises a number.

10. The blood pressure cuff of claim 7, wherein each sheet of the stack of sheets is sequentially marked.

11. The blood pressure cuff of claim 10, wherein each sheet of the stack of sheets is sequentially marked with a number.

12. The blood pressure cuff of claim 10, wherein each sheet of the stack of sheets is sequentially marked with a letter.

13. The blood pressure cuff of claim 10, wherein each sheet of the stack of sheets is sequentially marked with a color, where each sequential sheet has a darker color than a preceding sheet.

14. The blood pressure cuff of claim 7, wherein the stack of sheets has a partial cut located at a back section of each sheet, whereby a user of the invention can easily tear away individual sheets.

15. The blood pressure cuff of claim 7, wherein each sheet of the stack of sheets has a base section and a corner section, where the base section comprises an adhesive substance that enables the base section to removably adhere to an adjacent sheet, and where the corner section lacks any such adhesive substance, thereby allowing a user of the invention to easily select only the uppermost sheet for removal.

16. The blood pressure cuff of claim 7, where the sheets of the stack of sheets are stapled together, whereby a user can easily remove a single sheet from the stack of sheets.

17. The blood pressure cuff of claim 16, where the stack of sheets is stabled to the flat portion of the cover.

18. A method of operating a blood pressure cuff comprising the steps of
obtaining a blood pressure cuff, where the blood pressure cuff comprises a main bladder and a cover, where the cover comprises a stack of sheets and a base, where the base comprises a flat portion of thin material and a plurality of tabs, where each tab comprises a section of thin material that extends away from the flat portion, where the stack of sheets comprises a plurality of thin, film member sheets, where the plurality of sheets are removably secured together such that each sheet may easily detach from an adjacent sheet, where the cover is removably secured to the main bladder using the plurality of tabs;
removing one of the sheets from the stack of sheets; and
placing the blood pressure cuff around an appendage of a patient, whereby the stack of sheets creates a barrier between the blood pressure cuff and the patient.

19. The method of claim 18, where each tab of the base comprises an adhesive covering at least a portion of its surface, where the adhesive secures the tab to the main bladder.

20. The method of claim 18, further comprising the steps of
removing the cover from the main body; and
securing another cover to the main body, where the additional cover comprises a stack of sheets and a base, where the base comprises a flat portion of thin material and a plurality of tabs, where each tab comprises a section of thin material that extends away from the flat portion, where the stack of sheets comprises a plurality of thin, film member sheets, where the plurality of sheets are removably secured together such that each sheet may easily detach from an adjacent sheet, where the additional cover is removably secured to the main bladder using the plurality of tabs.
